(19) United States
(12) Patent Application Publication
(10) Pub. No.: US 2016/0024469 A1
(43) Pub. Date: Jan. 28, 2016

(54) Non-conventional Cellular Based Immunotherapy

(71) Applicant: Allan Yang Wu, Cathedral City, CA (US)

(72) Inventor: Allan Yang Wu, Cathedral City, CA (US)

(21) Appl. No.: 14/341,853

(22) Filed: Jul. 28, 2014

(51) Int. Cl. C12N 5/078; A61K 35/14; C12N 13/00

(52) U.S. Cl. CPC C12N 5/0634; C12N 13/00; A61K 35/14; C12N 2500/84

(57) ABSTRACT

The proposed patent is a method of immunotherapy wherein unique and novel sources of antigen generating organisms/viruses/cells are incubated or electroporated into further novel immunogenic cells. This method may be used for cancer therapy or therapy of benign disease. This method may also be used for immunization from infectious organisms such as bacteria, fungi, viruses or parasites. An aliquot of cells or materials obtained from the antigen generating component or electroporated immunogenic cells may be preserved long-term (i.e. cryopreservation or lyophilization or desiccation) for the purpose of forming a library or archive for future therapy or drug development/research.

Most importantly non-obviousness of this patent is clearly elucidated in the proposed method of immunotherapy whereby cancer stem cells (either directly isolated in culture or created by an induced process like iPSCs) are lysed for their protein antigens and electroporated or incubated with novel immunogenic cells (not dendritic cells) such as SVF and given back to the same patient in an autologous and personalized fashion. (In this way both MHC I and MHC II pathways may be utilized and a plurality of non-dendritic cells with antigen presenting capabilities are advantageously used in a plural immune like fashion raising immunogenicity through multiple novel and unique epitopes.

### CROSS REFERENCE TO RELATED APPLICATIONS

This invention claims benefit of priority to U.S. provisional patent application Ser. No. 61/958,486 filed Jul. 29, 2013.

### TECHNICAL FIELD

The invention relates generally to methods and systems for the delivery of manufactured therapeutic cells ex-vivo, which are eventually transferred in-vivo for the purpose of immunotherapy.

### DESCRIPTION OF PRIOR ART

Immunotherapy in the context of cellular therapy for cancer can be performed by transfecting expression vectors within dendritic cells to enhance the probability of antigen presentation and activate precise immune activity against any cell expressing the antigen such as a cancerous or unwanted cell or tissue. A good example of this is seen in the treatment of prostate cancer, in which vectors encoding modified prostate antigens may be transfected into dendritic cells (WO 2001074855 A3). This process, though efficacious, is time consuming and requires multiple complex steps in manufacturing. Furthermore, the process only takes advantage of the MHC II pathway of immunogenicity formation. This issue is addressed in EP 1565196 B1, wherein quick electroporation of antigen into only dendritic cells is explained, but no use of the MHC I pathway or electroporation into any type of stem cell or even use of cancer stem cells (as a source of antigen) is explained or claimed. Conversely, US 20110313229 A1 does mention use of cancer stem cells as a source of material for antigen for immunotherapy, however, there is no mention of induced pluripotent stem cells (iPSCs), nor is there mention of electroporation as a mode of transfecting cell lysate material into antigen presenting cells. (Electro-fusion of cancer stem cells and antigen presenting cells (APCs) is described but no discussion of electroporation of cancer stem cell antigen into APCs is found.)

For this reason a facile and inexpensive method to manufacture an autologous (and even allogeneic) and personalized cellular immunotherapy would be of great utility if the technical hurdles presented above are taken into consideration as in this patent presented.

### SUMMARY OF THE INVENTION

The invention is a method of immunotherapy for the treatment of benign and/or malignant tissues. The method is also capable of providing immunization to bacterial/viral/fungal/parasitic infections. Lysates from various biologic sources may be introduced into either a heterogenous or discreet population of immune cells by a facile process of electroporation. In this way the internalized antigens are processed by the immune cells to undergo either MHC I and/or MHC II pathways of cell mediated antigen presentation. This ultimately will lead to “recognition” of the antigens as “foreign” and urge the host immune system to attack the cells/organism/pathogen expressing the antigen. What is unique to this patent is the source of immune cells and source of antigen. Prior art in this field has solely focused on pure populations of dendritic cells. The current patent instead utilizes a plurality of immune cells generated from either bone marrow aspiration, peripheral blood collection, placental cells, umbilical cord cells or isolation of stromal vascular fraction from lipoaspiration. (These sources of immune cells can optionally be further isolated for discreet fractions of cells other than dendritic cells for use in the method.) The current patent also utilizes a unique source of antigen cells, in particular, iPSCs, which may be derived from correlated tissues of interest. For example if a patient had pancreatic cancer, iPSC cells can be developed from pancreatic cells and grown in culture for eventual lysate processing and electroporation into the immune cells.

### EXAMPLE 1

### Autologous Immunotherapy for Endometriosis

A patient with refractory endometriosis was placed under general anesthesia, after which 50 cc of fat was removed from the abdomen by tumescent liposuction and stromal vascular fraction (SVF) further separated from the fat by method of collagenase digestion (or sonication) and then centrifugation. The immune cells were placed aside in a sterile container and resuspended in a suitable physiologic buffer or medium.

Next, a dilation and curettage was obtained from the uterine cavity. This biopsy specimen was further processed to remove blood and enzymatically digested to break down the tissue into cellular components and washed and then centrifuged and resuspended in a suitable physiologic buffer or medium. The purified biopsy cells may then be further processed with a lysis buffer or direct sonication to create a whole cell lysate to release antigens specific for endometrial tissue. (Optionally, protein from the whole cell lysate may be further processed and purified and used for electroporation into immune cells.)

Finally the whole cell lysate is mixed with the SVF cells and electroporation is used to force antigen into the SVF cells. The SVF cells are then transfused intravenously into the same patient in the same procedural setting. (Optionally, the electroporated SVF cells can be given percutaneously through a needle into diseased tissue or intramuscularly or intraperitoneally or intralymphatically.)

### EXAMPLE 2

### Autologous Immunotherapy for Pancreatic Cancer

A patient with pancreatic cancer received a surgical resection which contained a 2 cm margin of healthy pancreatic tissue. Sterile biopsies from healthy and disease pancreatic tissue was obtained after pathology diagnosis was obtained. Specimens were enzymatically digested to release cells. (Enzyme is deactivated after cells are released.) In the same (or previous or separate) surgical setting liposuctioned fat was obtained from the same patient and SVF was isolated by collagenase digestion or sonication, then resuspended in an appropriate physiologic buffer/media in a sterile container.

Pancreatic tumor cells were then lysed by sonication or lysis buffer. Tumor whole cell lysate was mixed with SVF cells and electroporation was used to introduce pancreatic tumor antigen into the cells. The electroporated SVF cells were re-introduced into the same patient for immuno-cancer therapy.

Optionally, iPSCs may be derived from either normal pancreatic cells or pancreatic tumor cells from the same patient. (Pancreatic tumor cells are already immortalized, but they may not be dedifferentiated and express unique antigens associated with pancreatic tumor stem cells.) These cells may then function as a source of antigen for whole cell lysates that are ultimately electroporated into immune cells or SVF as above.

### BRIEF DESCRIPTION OF DRAWINGS

FIG. 1: Proposed method of novel immunotherapy.

### DETAILED DESCRIPTION OF DRAWINGS

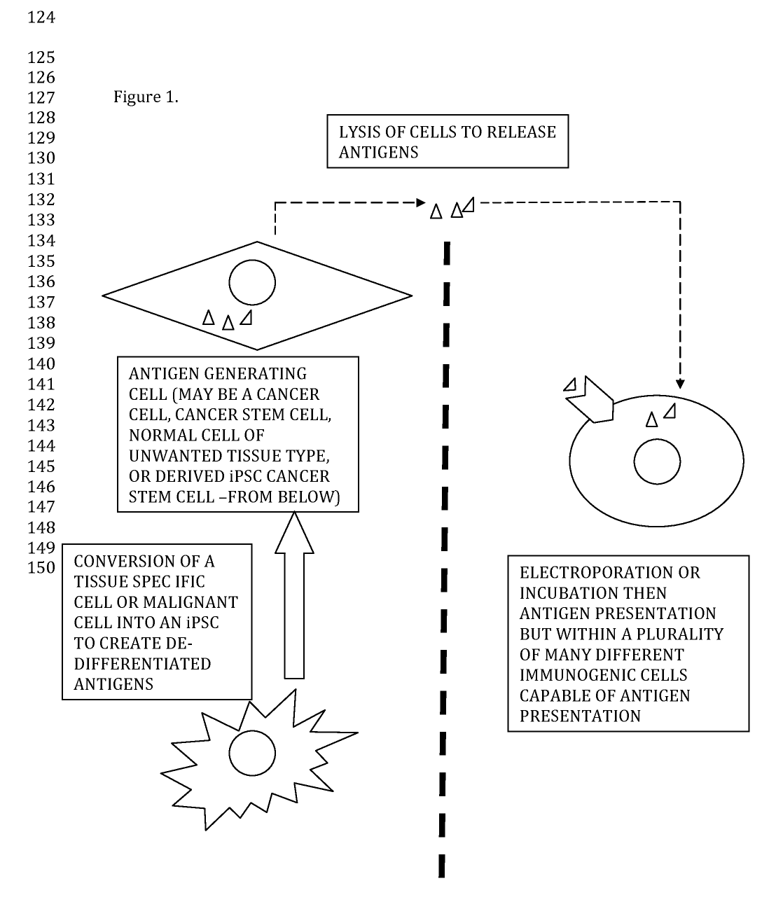

FIG. 1: Proposed method of novel immunotherapy wherein: left of heavy dotted line are the antigen generating cells. The upper left cell (spindle shaped) may be a tumor cell or cell/tissue type that is unwanted, or it may be a derived iPSC from (from the bottom left cell-stellate shaped). The antigen generating cell is lysed (dotted arrow) and antigens (small triangles) ultimately introduced into the immunogenic cell (right of heavy dotted line) and are ultimately presented by the immunogenic cell (Y-shaped object holding triangle on surface of cell to right of heavy dotted line).

I claim:

1. A method of creating whole cell lysate derived from antigen generating cells which may be comprised of either:
a. benign tumor cells,
b. malignant tumor cells,
c. benign differentiated adult tissue,
d. induced pluripotent stem cells (iPSCs)derived from tumor cells,
e. iPSCs derived from differentiated adult tissue corresponding to tumor type,
f. iPSCs derived from fibroblasts,
g. iPSCs derived by a process with or without Yamanaka factors or an optimal combination of overexpression of the following genes: Sox1, Sox2, Sox3, Sox15, Sox18, Klf1, Klf5, Klf2, Klf4, N-myc, L-myc, c-myc, Nanog, LIN28, Glis1 by way of vector transfection, small molecule induction or RNA or microRNA based methods
h. cultured tumor stem cells,
i. resistant tumor cells in vitro derived by cell culture,
j. viral or bacterial or fungal or parasitic cultures,
k. or any optimal mix of the above
and using said lysate to be incubated or electroporated with/into immunogenic cells derived from:
a. the stromal vascular fraction (SVF) of adipose tissue,
b. a fraction or component of SVF,
c. the stromal vascular fraction (SVF) of omental tissue,
d. adipose derived stem cells (ADSC),
e. bone marrow,
f. bone marrow stem cells,
g. induced pluripotent stem cells,
h. placental stem cells,
i. a fraction or component of bone marrow,
j. a fraction or component of placental tissue,
k. peripheral blood leukocytes other than dendritic cells,
l. fraction or component of blood leukocytes other than dendritic cells,
m. induced pluripotent stem cells
n. or any optimal mix of the above
for the purpose of cellular immunotherapy in the treatment of a human (autologous and/or allogeneic) or animal (autologous and/or allogeneic) for:
a. cancer or
b. unwanted benign tissues or
c. immunization against foreign pathogens
and the immunogenic cells may be introduced back into the host or recipient:
a. intravenously,
b. intraperitoneally,
c. intralesional injection,
d. intramuscular,
e. intralymphatic,
f. intrathecal,
i. subdural,
j. intracranial,
k. intraoccular,
l. intra-articular (within joints),
m. intradermal,
n. or any combination of the above.

2. An aliquot of antigen generating cells or electroporated immunogenic cells may be preserved long-term (i.e. cryopreservation or lyophilization or desiccation) for the purpose of forming an archival library or source of future therapy or further research to develop new forms of therapeutic drugs.

3. The above method in claim 1, wherein either:
a. Gamma Interferon,
b. Interleuken 6,
c. transporter associated with antigen processing (TAP),
d. TAP1,
e. TAP2,
f. tapasin,
g. calreticulin,
h. calnexin,
i. Erp57,
j. TNF (and subtypes),
k. or any optimal mix of the above
may be added to any of the processing techniques to enhance immunogenicity.
